(19) United States
(12) Patent Application Publication
(10) Pub. No.: US 2024/0425894 A1
(43) Pub. Date: Dec. 26, 2024

(54) CELL CULTURE PROCESSES

(71) Applicant: UCB BIOPHARMA SRL, BRUSSELS (BE)

(72) Inventors: BASSEM BEN YAHIA, BRUSSELS (BE); ANTOINE PHILIPPE THOMAS PIEDNOIR, BRUSSELS (BE)

(21) Appl. No.: 18/287,057

(22) PCT Filed: Apr. 13, 2022
(86) PCT No.: PCT/EP2022/059903

§ 371 Date: Oct. 16, 2023

(30) Foreign Application Priority Data
Apr. 16, 2021 (GB) 2105424.2

(51) Int. Cl. C12P 21/02; C12N 5/00

(52) U.S. Cl. CPC C12P 21/02; C12N 5/0018; C12N 2500/32

(57) ABSTRACT

The present invention belongs to the field of the manufacture of recombinant proteins, particularly antibodies. More specifically, it relates to cell culture processes for expressing proteins with improved yield during commercial scale manufacturing.

### CROSS-REFERENCE TO RELATED APPLICATION

This application is the U.S. national stage application of International Patent Application No. PCT/EP2022/059903, filed Apr. 13, 2022.

### FIELD OF INVENTION

The present invention belongs to the field of the manufacture of recombinant proteins, particularly antibodies. More specifically, it relates to cell culture processes for expressing proteins with improved yield during commercial scale manufacturing.

### BACKGROUND OF THE INVENTION

The development of recombinant proteins as therapeutic proteins, such as therapeutic antibodies, requires production of the recombinant proteins at an industrial scale. In order to achieve this, different expression systems, both prokaryotic and eukaryotic systems, may be employed. Over the past two decades, however, the majority of the proteins approved as therapeutic have been manufactured through mammalian cell cultures and such system remains the preferred expression system for producing large quantity of recombinant proteins for human use.

Over the last 30 years, much effort has been dedicated to establishing the basic parameters of cell culture and recombinant protein expression with much focus of the research dedicated to reaching optimal cell growth through changes of the composition of the cell culture media (see e.g. Hecklau C. et al., 2016; Zang Li. et al., 2011) and operating conditions and, development of large bioreactors.

Whilst yield is still a very important aspect of mammalian cell culture, in recent years the focus has shifted towards controlling product quality and process consistency at all stages of development and production scale. Therapeutic proteins produced by mammalian cell culture exhibit varying levels of heterogeneity. Such heterogeneity includes, but is not limited to, different glycosylation patterns, differences resulting from deamidation or oxidation, different charge or size variants. In recent years, there has been a steady trend toward subcutaneous delivery of therapeutic proteins which requires formulating therapeutic proteins at high concentrations. High concentrations have been associated with increased aggregate levels (Purdie J. et al., 2016). Increased charge variants, such as increased levels of acidic species may affect the protein stability (Banks D. D. et al., 2009). Cell culture conditions, such as the composition of the medium (Kshirsagar R. et al., 2012; US20130281355; WO2013158275; Ben Yahia B. et al., 2016; Ben Yahia B. et al., 2016) and the growing conditions, including feeding strategy (WO2018219968; Pan et al., 2017), pH and temperature (U.S. Pat. No. 8,765,413) have been shown to impact the yield and the quality attributes of therapeutic proteins.

Yet, there remains the need to provide cell culture methods with improved yield for the production of therapeutic proteins, while having a minimal impact on protein heterogeneity.

### SUMMARY OF THE INVENTION

In a first aspect, the invention provides a process for culturing mammalian cells expressing a recombinant protein, comprising the steps of: culturing said mammalian cells in a culture medium and adding between day 1 and day 7 of the culture an exceptional bolus of cysteine (Cys), tryptophan (Trp) and tyrosine (Tyr), wherein said exceptional bolus provides high concentrations of Cys, Tyr and Trp in the cell culture. In a second aspect, the invention provides a process for producing a recombinant protein, wherein the process comprises the steps of: culturing mammalian cells expressing said recombinant protein in a culture medium and adding, between day 1 and day 7 of the culture, an exceptional bolus of cysteine (Cys), tryptophan (Trp) and tyrosine (Tyr), wherein said exceptional bolus provides high concentrations of Cys, Tyr and Trp in the cell culture.

In a third aspect, the invention provides a process for increasing specific productivity (Qp) of mammalian cells in culture, wherein the mammalian cells express a recombinant protein, comprises the steps of: culturing the mammalian cells in a culture medium and adding between day 1 and day 7 of the culture an exceptional bolus of (Cys), tryptophan (Trp) and tyrosine (Tyr), wherein said exceptional bolus provides high concentrations of Cys, Tyr and Trp in the cell culture.

In the context of any one of these aspects, the total concentrations of Cys, Trp and Tyr present in the cell culture upon addition of the exceptional bolus are respectively: at least about 2.45 mM for Cys; at least about 1.50 mM for Trp; and at least about 2.75 mM for Tyr.

In a further preferred embodiment of any of these aspects, the total concentrations of Cys, Tyr and Trp, are controlled in order to reach at least about 2.45 mM, at least about 2.75 mM and at least about 1.50 mM, respectively, in the cell culture at least one day before the maximum viable cell concentration peak is reached.

Preferably, these high concentrations are reached simultaneously or sequentially. Cys, Tyr and Trp can be part of the same feed medium or of different feed media.

### Definitions

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the term “and/or” used in a phrase such as “A and/or B” herein is intended to include “A and B”, “A or B”, “A”, and “B”.

As used in the specification and claims, the term “cell culture” or “culture” is meant the growth and propagation of cells in vitro, i.e. outside of an organism or tissue. Suitable culture conditions for mammalian cells are known in the art, such as taught in Ozturk & Hu (2005). Mammalian cells may be cultivated in suspension or while attached to a solid substrate.

The terms “cell culture medium,” “culture medium”, “medium,” and any plural thereof, refer to any medium in which cells of any type can be cultivated. A “basal medium” refers to a cell culture medium that contains all of the essential ingredients useful for cell metabolism. This includes for instance amino acids, lipids, carbon source, vitamins and mineral salts. DMEM (Dulbeccos' Modified Eagles Medium), RPMI (Roswell Park Memorial Institute Medium) or medium F12 (Ham's F12 medium) are examples of commercially available basal media. Other suitable media have been described for instance in WO98/08934 and US2006/0148074 (both incorporated herein in their entirety). Further suitable commercially available media include, but are not limited to, AmpliCHO CD medium, Dynamis™ Medium, EX-CELL® Advanced™ CHO Fed-batch System, CD FortiCHO™ medium, CP OptiCHO™ medium, Minimum Essential Media (MEM), BalanCDR CHO Growth A Medium, ActiPro™ medium, DMEM-Dulbecco's Modified Eagle Medium and RPMI-1640 medium. Alternatively, said basal medium can be a proprietary medium, also herein called “chemically defined medium” or “chemically defined culture medium”, in which all of the components can be described in terms of the chemical formulas and are present in specific concentrations. The culture medium is preferably free of proteins and free of serum and can be supplemented by any additional compound(s) such as amino acids, salts, sugars, vitamins, hormones, growth factors, depending on the needs of the cells in culture.

The term “feed medium” (and plural thereof) refers to a medium used as a supplementation during culture, in fed-batch mode, to replenish the nutrients which are consumed. The feed medium can be a commercially available feed medium or a proprietary feed medium. Suitable commercially available feed media include, but are not limited to, Cell Boost™ supplements, EfficientFeed™ supplements, ExpiCHO™ Feeds. Alternatively, said feed medium can be a proprietary feed medium, also herein called “defined feed medium” or “chemically defined feed medium”, in which all of the components can be described in terms of the chemical formulas and are present in specific concentrations. A feed medium is typically concentrated in order not to increase to a high level the total volume of the culture. Such a feed medium can contain most of the components of the cell culture medium at, for example, about 1.5×, 2×, 5×, 6×, 7×, 8×, 9×, 10×, 12×, 14×, 16×, 20×, 30×, 50×, 100×, 200× or even 500× of their normal amount in a basal medium. Proprietary feed media are typically in powder. Commercial feeds are either liquid or in powder. When feeds are already in liquid form, they are used as such, according to the leaflet. Feeds which are in powder need to be solubilised, in water for instance, before use. They are supposed to be solubilised in a given amount of water (e.g. 100 g in 1 L of water, see FIG. 1A). However, feeds in powder can be further concentrated. In such a case, they will be solubilised using less liquid than normally needed for said quantity (e.g. 200 g in 1 L of water, see FIG. 1B). Liquid commercial feeds or feeds in powder that are prepared according to the standard protocol are herein also called “normal” feed. Liquid commercial feeds or feeds in powder that are prepared according to a concentrated process are herein called “concentrated feed” or “concentrated main feed”.

Different feed media of different compositions can be added throughout the culture process. For instance, three different feed media can be used during the same process: one feed medium consisting of the carbon source (e.g. glucose), one feed medium comprising most of the nutrients which are consumed (this feed is also named main feed medium), and a further feed medium comprising some further nutrients for instance when these nutrients present aggregation/stability issues.

The term “bioreactor” refers to any system in which cells can be cultivated. It includes but is not limited to flasks, static flasks, spinner flasks, tubes, shake tubes, shake bottles, wave bags, bioreactors, fibre bioreactors, and stirred-tank bioreactors with or without microcarriers. Alternatively, this term also includes microtiter plates, capillaries or multi-well plates. Any size of bioreactor can be used, for instance from 1 millilitre (1 mL, very small scale) to 20000 litres (20000 L or 20 KL, very large scale), such as 1 mL, 5 mL, 0.01 L, 0.1 L, 1 L, 2 L, 5 L, 10 L, 50 L, 100 L, 500 L, 1000 L (or 1 KL), 2000 L (or 2 KL), 5000 L (or 5 KL), 10000 L (or 10 KL), 15000 L (or 15 KL) or 20000 L (20 KL).

The term “fed-batch culture” refers to a method of culturing cells, where there is a bolus (typically several bolus) or continuous feed medium (or feed media) supplementation to replenish the nutrients which are consumed, without removal of any medium. Feed(s) can be added according to a predetermined schedule of, for example, every day, once every two days, once every three days, etc. Alternatively, should the feeding be continuous, the feeding rate can be varied throughout the culture. This cell culture technique has the potential to obtain high cell densities in the order of greater than 10×10⁶ to 30×10⁶ cells/ml, depending on the media formulations, cell line, and other cell growth conditions. A biphasic culture condition can be created and sustained by a variety of feed strategies and media formulations.

Alternatively, a perfusion culture can be used. Perfusion culture is one in which the cell culture receives fresh perfusion feed medium while simultaneously removing spent medium. Perfusion can be continuous, step-wise, intermittent, or a combination of any or all of any of these. Perfusion rates can be less than a working volume to many working volumes per day. Preferably the cells are retained in the culture and the spent medium that is removed is substantially free of cells or has significantly fewer cells than the culture. Perfusion can be accomplished by a number of cell retention techniques including centrifugation, sedimentation, or filtration (see for example Voisard et al., 2003). When using the processes, methods and/or cell culture techniques of the present invention, in mammalian cells, the recombinant proteins are generally directly secreted into the culture medium. Once said protein is secreted into the medium, supernatants from such expression systems can be first clarified, in order to start isolating the protein of interest and concentrate if before it is purified and formulated.

The term “production phase” according to the present invention comprises that stage of cell culturing during the process for manufacturing a recombinant protein when the cells express (i.e. produce) the recombinant polypeptide(s). The production phase begins when the titre of the desired product increases and ends with harvest of the cells or the cell culture fluid or supernatant. Typically, at the beginning of the production phase, the cell culture is transferred to a production vessel, such as a bioreactor. Harvest is the step during which the cell culture fluid is removed from the production vessel, in order for the recombinant protein (such as. the recombinant antibody), to be recovered and purified in subsequent steps.

“Cysteine”, is an amino acid having a molecular weight of 121.16 g/mol. The L enantiomer is preferred (i.e. L-cysteine). The term also encompassed any salts or derivatives thereof, such as (but not limited to) cysteine hydrate, cysteine dihydrate, cysteine hydrochloride, cysteine dihydrochloride, cysteine hydrochloride monohydrate, cysteine S-sulfate (also known as S-sulfocysteine), acetylcysteine, N-acetylcysteine. Alternatively, any cysteine/cystine analogs disclosed in WO2019106091 can be used.

“Cystine” is an amino acid having a molecular weight of 240.3 g/mol. The L enantiomer is preferred (i.e. L-cystine). The term also encompassed any salts or derivatives thereof, such as cystine hydrochloride, cystine dihydrochloride, N,N′-Diacetyl-L-cystine, N, N′-diacetyl-L-cystine dimethyl ester or L-Cystine dimethyl ester.

“Cysteine” and “cystine” in the cell culture medium are in constant equilibrium wherein two molecules of cysteine oxidize into a molecule of cystine which reduces back to two molecules of cysteine. Although within this document it is mainly referred to cysteine (alternatively it is referred to Cys), for ease of reading, there is no limitation to cysteine. Therefore, the term “Cys” refers to cysteine, cystine, salts thereof, derivatives thereof or any combination thereof. For instance, when it is referred to “About 2.45 mM for Cys”, the skilled person understands that it encompasses about 2.45 mM of L-cysteine, L-cystine, salts thereof, derivatives thereof or combinations of cysteine and cystine for instance. When expressed in g/L, “about 2.45 mM of Cys” corresponds for instance to about 0.30 g/L of L-cysteine or about 0.60 g/L of L-cystine.

“Tyrosine”, also herein referred to as “Tyr”, is an amino acid having a molecular weight of 181.19 g/mol. The L enantiomer is preferred (i.e. L-tyrosine). The term also encompassed any salts or derivatives thereof, such as (but not limited to) tyrosine disodium salt, tyrosine disodium hydrate, tyrosine disodium dihydrate, N-Acetyl-L-tyrosine or phosphotyrosine sodium. When expressed in g/L, “about 2.75 mM of Tyr” corresponds for instance to about 0.50 g/L of L-tyrosine.

“Tryptophan”, also herein referred to as “Trp”, is an amino acid having a molecular weight of 204.23 g/mol. The L enantiomer is preferred (i.e. L-tryptophan). The term also encompassed any salt thereof, such as (but not limited to) tryptophan sodium. When expressed in g/L, “about 1.50 mM of Trp” corresponds for instance to about 0.30 g/L of L-tryptophan.

The term “high concentration” for any one of Cys, Trp or Tyr refers to a concentration at or above 2.0 mM for Cysteine (or cystine or any salt thereof), at or above 1.7 mM for Tyr or at or above 1 mM for Trp (see for instance Pan et al., 2017).

As used herein, “cell concentration” (also known as “cell density”) refers to the number of cells in a given volume of culture medium.

The term “Viable cell concentration” (or “VCC”) refers to the number of living cells in a given volume of culture medium. This is determined by standard viability assays. It should be understood that the skilled person knows how to determine the maximum VCC for each specific cell line: this is typically performed thanks to one or more initial experiments. Once the day when the maximum VCC is reached is known for one cell line expressing a given protein under given conditions, a process according to the invention can be designed. There is no need to determine VCC for each and every experiment.

The term “IVCC” refers to the integral viable cell count and can be determined by finding the area under the cell culture growth curve (IVCC=∫₀^{t}VCC*dt).

The term “viability”, or “cell viability” refers to the ratio between the total number of viable cells and the total number of cells in culture. Although the viability is typically acceptable as long as it does not go below a 60% threshold compared to the start of the culture, the acceptable threshold can be determined on a case by case basis. Viability is often used to determine time for harvest. For instance, in fed-batch culture, harvest can be performed once viability reaches at least 60% or after about 14 days (typically 14 days+/−1 day) in culture. Standard methods can be used to determine the cell viability or VCC, such as via the use of the VI-CELL® XR automated cell counting device (Beckman-Coulter Inc.).

The term “titre” refers to the concentration of the protein of interest in solution. This is determined by standard titre assays, such as serial dilutions combined with a detection method (colorimetric, chromatographic etc.), with a CEDEX or protein A high-pressure liquid chromatography (HPLC), Biacore C® or ForteBIO Octet® methods, as used in the example section.

The term “specific productivity”, also known as “qp”, refers to the amount of protein of interest, produced per cell per day.

The term “higher titre” or “higher productivity”, and equivalents thereof, means that the titre or the productivity is increased by at least 10% when compared to the control culture condition. The titre or specific productivity will be considered as maintained if it is in the range of −10% to 10% compared to the control culture condition. The terms “lower titre” or “lower productivity”, and equivalents thereof, means that the titre or the productivity is decreased by at least 10% when compared to the control culture condition.

The term “heterogeneity” as used herein refers to differences between individual molecules, e.g. recombinant proteins, in a population of molecules produced by the same manufacturing process, or within the same manufacturing batch. Heterogeneity can result from incomplete or inhomogeneous modifications of the recombinant polypeptides, e.g. due to post-translational modifications of the polypeptide or to misincorporation during transcription or translation. Post-translational modifications can e.g. be the result of deamination reactions and/or oxidation reactions and/or covalent addition of small molecules such as glycation reactions and/or isomerization reactions and/or fragmentation reactions and/or other reactions and also include variation on the glycation patterns. The chemo-physical manifestation of such heterogeneity leads to various characteristics in the resulting recombinant polypeptide preparations which include, but are not limited to, charge variant profile, colour or colour intensity and molecular weight profile.

The reduction of the charge heterogeneity is preferably defined by measuring the acidic peak group (APG) species in the population of recombinant proteins produced in the cell culture. A possible way to measure the APG reduction, is by determining via Imaged Capillary Electrophoresis (e.g. ProteinSimple iCE3) the relative percentage of acidic (APG for Acidic Peak Group) isoforms of the recombinant proteins produced in a cell culture medium with or without the cysteine/cysteine analogs, which recombinant protein is at time of measurement preferably purified. When measuring the isoforms of the recombination proteins, besides the APG also the basic isoforms (Basic Peak Group (BPG)) and the main charge species are measured, wherein the main charge species represents the isoform of the recombinant protein that one wishes to obtain. It is preferred that when the APG is decreased, there is substantially no increase of the BPG. Preferably, when the APG is decreased, the main charge species level increases.

The term “protein” as used herein includes peptides, polypeptides and proteins and refers to compound comprising two or more amino acid residues. A protein according to the present invention includes but is not limited to a cytokine, a growth factor, a hormone, a fusion protein, an antibody or a fragment thereof. A therapeutic protein refers to a protein that can be used or that is used in therapy.

The term “recombinant protein” means a protein produced by recombinant technics. Recombinant technics are well within the knowledge of the skilled person (see for instance Sambrook et al., 1989, and updates).

The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old-World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDRs; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments, more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbeR) or Fab-(dsscFv)2 (also referred to as TrYbe®, see WO2015197772 for instance). Antibody fragments as defined above are known in the art.

### DETAILED DESCRIPTION OF THE INVENTION

This disclosure relates to processes for the production of recombinant proteins in mammalian cells and of culturing mammalian cells expressing a recombinant protein. In particular, the invention is based on the finding from the inventors that by controlling 1) the amounts of cysteine (Cys), tryptophan (Trp) and tyrosine (Tyr) brought by the feed medium (or feed media) and 2) the timing of addition, there are surprising effects of reduction of cell growth while increasing specific productivity of the cell and increasing production yield without impacting the quality of the recombinant proteins. In particular, the formation of product-related variants—such as charge variants, deaminated variants or oxidized species—is reduced by implementation of the processes described herein.

The processes herein described rely on the control of the concentrations of Cys, Trp and Tyr, in the cell culture, during the production of the recombinant protein of interest. It was indeed surprisingly found that by adding an exceptional bolus feed comprising high and controlled concentrations of Cys, Tyr and Trp, in a timeline manner (such as one day to seven days after the start of the culture and/or at least one day prior to the day the VCC is expected to be reached), it was possible to reach higher concentration of each of these amino acids in the culture medium and to increase specific monoclonal antibody productivity of the cells in culture.

This disclosure describes how to control these parameters in order to maximize the production bioreactor yield, minimize the cell growth and minimize the micro heterogeneity of the recombinant protein produced. This disclosure provides specific examples of processes (in particular fed-batch processes) in which these parameters are controlled within the claimed ranges and provides specific examples of possible modes of addition of cysteine, tyrosine and tryptophan (exceptional bolus and its timings, versus continuous/semi-continuous etc.).

In one embodiment, the invention provides a process for culturing mammalian cells expressing a recombinant protein, comprising the steps of: culturing said mammalian cells in a culture medium and adding between day 1 and day 7 of the culture an exceptional bolus of cysteine (Cys), tryptophan (Trp) and tyrosine (Tyr), wherein said exceptional bolus provides high concentrations of Cys, Tyr and Trp in the cell culture.

In another embodiment, the invention provides a process for producing a recombinant protein, wherein the process comprises the steps of culturing mammalian cells expressing said recombinant protein in a culture medium and adding, between day 1 and day 7 after the start of the culture, an exceptional bolus of cysteine (Cys), tryptophan (Trp) and tyrosine (Tyr), wherein said exceptional bolus provides high concentrations of Cys, Tyr and Trp in the cell culture.

In a further embodiment, herein described is a process for increasing specific productivity (Qp) of mammalian cells in culture, wherein the mammalian cells express a recombinant protein, comprises the steps of culturing the mammalian cells in a culture medium and adding between day 1 and day 7 of the culture an exceptional bolus of (Cys), tryptophan (Trp) and tyrosine (Tyr), wherein said exceptional bolus provides high concentrations of Cys, Tyr and Trp in the cell culture.

In the context of the invention as a whole, the processes for producing a recombinant protein, for culturing mammalian cells expressing a recombinant protein or for increasing the specific productivity of mammalian cells in culture, comprise the following main steps:
i) inoculating the mammalian cells in a culture medium in a bioreactor (such as a production bioreactor), wherein the culture medium optionally comprises an initial amount of cysteine/cystine, tyrosine and tryptophan
ii) culturing mammalian cells expressing the recombinant protein in a culture medium (alternatively named the cell culture),
iii) further adding to the cell culture:
   a. an exceptional bolus providing high concentrations of cysteine, tryptophan and tyrosine, in the cell culture, between day 1 and day 7 of the culture,
   b. a main feed at regular intervals to the cell culture,
   c. further bolus(es) of cysteine, tryptophan and tyrosine in order to prevent any depletion Cys, Tyr and Trp during the cell culture,
iv) optionally recovering the recombinant protein from the cell culture fluid (CCF).

Alternatively, the processes for producing a recombinant protein, for culturing mammalian cells expressing a recombinant protein or for increasing the specific productivity of mammalian cells in culture, comprise the following main steps:
(i) inoculating the mammalian cells in a culture medium in a bioreactor (such as a production bioreactor), wherein the culture medium optionally comprises an initial amount of cysteine/cystine, tyrosine and tryptophan,
(ii) progressing the culture through the production phase, according to a fed batch process, wherein the recombinant protein is produced by the mammalian cells, wherein, during said production phase, the cell culture is:
   a) supplemented, between day 1 and day 7 of the culture, with an exceptional bolus of cysteine (Cys), tryptophan (Trp) and tyrosine (Tyr), wherein said exceptional bolus provides high concentrations of Cys, Tyr and Trp in the cell culture,
   b) supplemented with at least one main feed medium at regular intervals to the cell culture, and
   c) further supplemented with one or more feed(s) of cysteine/cystine, tyrosine and tryptophan, in order to prevent any depletion Cys, Tyr and Trp during the cell culture,
iii) optionally recovering the recombinant protein from the cell culture fluid (CCF).

The skilled person would understand that the term “between day 1 and day 7 of the culture” means “one day to seven days after the start of the culture” or alternatively “one day to seven days after the inoculation of the mammalian cells in a bioreactor”. Indeed, the start of the culture or the day the mammalian cells are inoculated in a bioreactor corresponds to day 0. The skilled person would also understand that “between day 1 and day 7 of the culture” means anyone of day 1, day 2, day 3, day 4, day 5, day 6 or day 7 of the culture.

Cysteine/cystine, tyrosine and tryptophan may be added simultaneously or sequentially. When the three amino acids are added simultaneously, they can be provided as (a) all combined in a single solution, (b) in individual solutions each comprising one of Cys, Tyr or Trp; or (c) a combination of two amino acids in one solution and a second solution comprising the remaining amino acid. When the amino acids are added sequentially, they can be added in any order, preferably within a 2-hour window period (for instance if Cys is added at HO, Tyr can be added at H+1 and Trp at H+2, or if Tyr is added at HO, Trp can be added at H+0.5 and Cys at H+1.5).

In the context of the invention as a whole, high concentrations of Cys, Tyr and Trp are reached in the cell culture upon addition of the exceptional bolus of Cys, Tyr and Trp. In one aspect, the total concentrations of Cys, Trp and Tyr present in the cell culture upon addition of the exceptional bolus are respectively:
a. At least about 2.45 mM for Cys;
b. At least about 1.50 mM for Trp; and
c. At least about 2.75 mM for Tyr.

Alternatively, the total concentrations of Cys, Trp and Tyr present in the cell culture upon addition of the exceptional bolus are respectively:
a. From about 2.45 mM to about 6.6 mM for Cys;
b. From about 1.50 mM to about 2.9 mM for Trp; and
c. From about 2.75 mM to about 6.2 mM for Tyr.

The skilled person knows how to adapt the concentrations of Cys, Tyr and Trp of the exceptional bolus in order to reach these high total concentrations in the cell culture, upon addition of the exceptional bolus.

The processes according to the invention as a whole can further comprise a preliminary step of performing at least one initial experiment to determine the daily concentrations of Cys, Trp and Tyr in the culture medium after the start of the culture, in order to determine the amounts of Cys, Trp and Tyr to be added via an exceptional bolus, depending on the day said exceptional bolus is to be added. It has to be understood that this initial experiment does not need to be repeated each time the processes according to the invention are performed. In other words, once the amounts of Cys, Trp and Tyr to be added via an exceptional bolus are determined, in at least one initial experiment, for one specific clone, under given conditions, there is no need to control these concentrations each time the processes according to the invention are to be performed.

In the context of the processes according to the invention as a whole, the high concentrations of Cys, Tyr and Trp are preferably reached in the cell culture medium before the day the maximum VCC is reached (or before the day the maximum VCC is expected to be reached), upon addition of the exceptional bolus of Cys, Tyr and Trp. In one aspect, the total concentrations of Cys, Tyr and Trp present in the cell culture upon addition of the exceptional bolus, before the day the maximum VCC is reached (or before the day the maximum VCC is expected to be reached), consist of: at least about 2.45 mM for Cys; at least about 1.50 mM for Trp; and at least about 2.75 mM for Tyr. In another aspect, the total concentrations of Cys, Tyr and Trp present in the cell culture upon addition of the exceptional bolus, before the day the maximum VCC is reached (or before the day the maximum VCC is expected to be reached), consist of from about 2.45 mM to about 6.6 mM for Cys; from about 2.75 mM to about 6.2 mM for Tyr; and from about 1.50 mM to about 2.9 mM) for Trp. In a particular aspect, the concentration of Cys, Tyr and Trp are controlled in order to reach simultaneously (or sequentially) at least about 2.45 mM, at least about 2.75 mM and at least about 1.50 mM, respectively, in the cell culture medium, at least one day before the maximum VCC is reached (or is expected to be reached). These concentrations are reached thanks to the addition of an exceptional bolus of Cys, Tyr and Trp at least one day before the maximum VCC is reached (or is expected to be reached). In this context, the addition of Cys, Tyr and Trp preferably starts at the latest one day prior to the day the maximum VCC is reached (or is expected to be reached). Alternatively, the addition of Cys, Tyr and Trp preferably starts two, three, four, five, six or seven days prior to the day of maximum VCC (or prior to the expected day of maximum VCC). Even preferably, the addition of Cys, Tyr and Trp starts two, three or four days prior to the day of maximum VCC (or prior to the expected day of maximum VCC). For instance, should the maximum VCC be reached (or be expected to be reached) at day 8, the addition of Cys, Tyr and Trp can be performed at day 1, day 2, day 3, day 4, day 5, day 6 or day 7. In another example, should the maximum VCC be reached (or be expected to be reached) at day 5, the addition of Cys, Tyr and Trp can be performed at day 1, day 2, day 3 or day 4.

The processes according to the invention as a whole can also further comprise a preliminary step of performing at least one initial experiment to determine the day the maximum viable cell concentration (VCC) is reached for the mammalian cells in culture. It has to be understood that this initial experiment does not need to be repeated each time the processes according to the invention are performed. In other words, once the day the maximum VCC is reached (or is expected to be reached) is determined, in at least one initial experiment, for one specific clone, under given conditions, there is no need to control it each time the processes according to the invention are to be performed.

In the context of the invention as a whole, should any preliminary steps be performed (e.g. to determine the day the maximum viable cell concentration (VCC) or to determine the amounts of Cys, Trp and Tyr to be added via an exceptional bolus), the processes for producing a recombinant protein, for culturing mammalian cells expressing a recombinant protein or for increasing the specific productivity of mammalian cells in culture, comprise the following main steps:
i) optionally performing at least one initial experiment to determine the daily concentrations of Cys, Trp and Tyr in the culture medium after the start of the culture, in order to determine the amounts of Cys, Trp and Tyr to be added via an exceptional bolus, depending on the day said exceptional bolus is to be added,
ii) optionally performing at least one initial experiment to determine the day the maximum viable cell concentration (VCC) is reached for the mammalian cells in culture,
iii) inoculating the mammalian cells in a culture medium in a bioreactor (such as a production bioreactor), wherein the culture medium optionally comprises an initial amount of cysteine/cystine, tyrosine and tryptophan,
iv) culturing mammalian cells expressing the recombinant protein in a culture medium (alternatively named the cell culture),
v) further adding to the cell culture:
   a. an exceptional bolus providing high concentrations of cysteine, tryptophan and tyrosine, in the cell culture, between day 1 and day 7 of the culture,
   b. a main feed at regular intervals to the cell culture,
   c. further bolus(es) of cysteine, tryptophan and tyrosine in order to prevent any depletion Cys, Tyr and Trp during the cell culture,
vi) optionally recovering the recombinant protein from the cell culture fluid (CCF).

Alternatively, and should any preliminary steps be performed (e.g. to determine the day the maximum viable cell concentration (VCC) or to determine the amounts of Cys, Trp and Tyr to be added via an exceptional bolus, the processes for producing a recombinant protein, for culturing mammalian cells expressing a recombinant protein or for increasing the specific productivity of mammalian cells in culture, comprise the following main steps:

i) optionally performing at least one initial experiment to determine the daily concentrations of Cys, Trp and Tyr in the culture medium after the start of the culture, in order to determine the amounts of Cys, Trp and Tyr to be added via an exceptional bolus, depending on the day said exceptional bolus is to be added,
ii) optionally performing at least one initial experiment to determine the day the maximum viable cell concentration (VCC) is reached for the mammalian cells in culture,
iii) inoculating the mammalian cells in a culture medium in a bioreactor (such as a production bioreactor), wherein the culture medium optionally comprises an initial amount of cysteine/cystine, tyrosine and tryptophan,
iv) progressing the culture through the production phase, according to a fed batch process, wherein the recombinant protein is produced by the mammalian cells, wherein, during said production phase, the cell culture is:
   a) supplemented, between day 1 and day 7 of the culture, with an exceptional bolus of cysteine (Cys), tryptophan (Trp) and tyrosine (Tyr), wherein said exceptional bolus provides high concentrations of Cys, Tyr and Trp in the cell culture,
   b) supplemented with at least one main feed medium at regular intervals to the cell culture, and
   c) further supplemented with one or more feed(s) of cysteine/cystine, tyrosine and tryptophan, in order to prevent any depletion Cys, Tyr and Trp during the cell culture.
v) optionally recovering the recombinant protein from the cell culture fluid (CCF).

The inventors have shown that the important aspect of the invention, in particular to boost the specific productivity of the cells, is the exceptional bolus of Cys, Tyr and Trp, added in a timely manner, in order to reach specific minimal concentrations of these three amino acids, in the cell culture, as described herein. However, should this exceptional bolus be integrated in a production process already in place to produce a recombinant protein with already defined product quality attributes (PQA), in order not to risk a negative impact of high amounts of these amino acids on the PQA, it is recommended to keep the same total amounts of Cys, Tyr and Trp provided throughout the culture (compared to the process already in place). In such a case, the amount of Cys, Tyr and Trp in the additional boluses to be added after the exceptional bolus can be adapted.

Should it be necessary to remain within certain ranges of product quality attributes for one given recombinant protein expressed in one given mammalian cell line, the total amount of Cys, Tyr and Trp provided during the process preferably remain comparable to standard processes without exceptional bolus addition of Cys, Tyr and Trp (see FIG. 2). After addition of the exceptional bolus of Cys, Tyr and Trp is performed, the next addition of Cys, Tyr and Trp is then performed when the cumulative amount added of Cys, Tyr and Trp during the exceptional bolus is equivalent to the cumulative amount of a standard process without exceptional bolus addition of Cys, Tyr, and Trp in order to then fit with a standard process feeding strategy for the following days. For example, if the exceptional bolus is added at day 1 and the amount of Cys, Tyr and Trp added in the exceptional bolus corresponds to the amount of Cys, Tyr and Trp added during four days in a standard process, the feeding will resume on the fifth day. In another example, if the exceptional bolus is added at day 3 and the amount of Cys, Tyr and Trp added in the exceptional bolus corresponds to the amount of Cys, Tyr and Trp added during three days in a standard process, the feeding will resume on the sixth day. Similar strategy can be applied to produce a recombinant protein not having yet a certain ranges of product quality attributes to be fulfilled. In such a case, the skilled person would understand how to adapt a standard process, or a platform process, they are used to utilise to produce recombinant proteins.

Although within this document it is mainly referred to cysteine, for ease of reading, there is no limitation to cysteine itself, i.e. “cysteine” and “cystine” are interchangeable. As explained in the Definitions section, cysteine and cystine in the cell culture medium are in constant equilibrium wherein two molecules of cysteine oxidize into a molecule of cystine which reduces back to two molecules of cysteine. Should one prefers using cystine instead of cysteine, at least about 2.45 mM of cystine would have to be reached upon addition of the exceptional bolus. Alternatively, combinations of cysteine and cystine can be used. Further any salt thereof can be used.

In the context of the invention as a whole, Cys, Tyr and Trp are part of the same feed medium. Preferably the feed medium comprising Cys, Tyr and Trp does not contain any other nutrients (i.e. preferably the feed medium consists of Cys, Tyr and Trp). This implies that the other nutrients needed for the correct growth of the cells are brought via other feed media, such as the main feed medium. Alternatively, Cys, Tyr and Trp are part of different feed media, such as a first feed medium comprising or consisting of Cys, a second feed medium comprising or consisting of Tyr, and a third feed comprising or consisting of Trp. Other nutrients can be supplemented to satisfy cells demands either via one of these three feed media (should one of this feed media be a main feed) or are brought via at least a fourth feed medium.

In the context of the invention as a whole, between the start of the culture (day 0) and the addition of an exceptional bolus of a Cys, Tyr and Trp (at least on day before the maximum VCC is reached or no later than day 7), no Cys, Tyr and Trp are added. To the contrary, at least the quantities of Cys, Tyr and Trp that would have been added via daily bolus, or continuous feeding, according to standard feeding strategies are added at once as an exceptional bolus (between day 1 and day 7 of the culture and/or at least on day before the maximum VCC is expected to be reached), and then “normal” feeding strategy resumes, for instance when the cumulative amount added of Cys, Tyr and Trp during the exceptional bolus addition is equivalent to the cumulative amount of a standard process without exceptional bolus addition of Cys, Tyr, and Trp, in order to then fit with a standard process feeding strategy for the following days (See FIG. 2). For example, if the amount of Cys, Tyr and Trp added at day 1 in the exceptional bolus corresponds to the amount of Cys, Tyr and Trp added during four days in a standard process, the feeding will resume on the fifth day (see FIG. 2). It has to be understood that the day the maximum VCC is reached (or is expected to be reached) does not need to be controlled each time the processes according to the invention are performed. This day of the maximum VCC is indeed determined according to at least one preliminary experiment, as well-known by the skilled person. The above are non-limiting examples. Indeed, should one wish to keep the same total amount of Cys, Trp and Tyr added throughout the culture compared to a standard process, the skilled person will know how to adapt the global feeding strategy for Cys, Trp and Tyr, whatever the day of the exceptional bolus and whatever the day the normal feeding resumes (i.e. one day or more after the exceptional bolus). As a non-limiting example, should one wish to provide total amounts of Cys, Tyr and Trp of 5 mmol per litre of cell culture volume throughout a 10-day-long culture, including 0.5 mM of each amino acid provided in the basal medium, one could design a process in which an exceptional bolus is added on day 2, providing 2.45 mM of Cys, 1.50 mM of Trp, 2.75 mM of Tyr in the cell culture, and then further feeds are added on day 4, day 6 and day 8, each feed providing respectively 0.85 mM of Cys, 1.17 mM of Trp, 0.75 mM of Tyr in the cell culture. In another non-limiting example, should one wish to provide total amounts of Cys, Tyr and Trp of 10 mmol per litre of cell culture volume throughout a 14-day-long culture, including 1 mM of each amino acid provided in the basal medium, and considering a maximum VCC on day 6, one could design a process in which an exceptional bolus is added on day 4, providing 3 mM of Cys, 2 mM of Trp, 3 mM of Tyr in the cell culture and then further feeds are added daily from day 7 to day 12, each feed providing respectively 1 mM of Cys, 1.17 mM of Trp, 1 mM of Tyr in the cell culture.

It should be understood that the skilled person knows how to:
Determine the day the maximum VCC is reached for each specific cell line/clone: this is typically performed thanks to one or more initial experiments. Once the day when the maximum VCC is reached (or is expected to be reached) is known for one cell line expressing a given protein under given conditions, a process according to the invention can be designed. There is no need to determine VCC for each and every experiment or batch.
Measure the concentrations of cysteine (and/or cystine), tyrosine and tryptophan added to and/or present in a cell culture at a specific phase, such as the production phase. Such determination is typically performed thanks to one or more initial experiments. For example, this can be done as described in the Examples herein (although not limited to these methods). Once the respective amounts are known for one cell line expressing a given protein under given conditions, a process according to the invention can be designed. There is no need to determine the amounts of cysteine (and/or cystine), tyrosine and tryptophan for each and every experiment or production batch.
Measure the total amount of recombinant protein produced by one cell line expressing a given protein under given conditions, and consequently apply the teaching of the present invention to achieve the desired technical effect. This can also be done as described in the Examples herein, such as using a ForteBio Octet model analyser (ForteBio, Inc.) or protein A high-pressure liquid chromatography (HPLC) with cell culture supernatant samples which were stored at −80° C. prior to analysis. Once the total amount of recombinant protein produced by a cell culture is known for one cell line expressing a given protein under given conditions, a process according to the invention can be designed. There is no need to determine such total amount for each and every experiment or batch.

In the context of the invention as a whole, the culture medium at the start of the culture (alternatively herein named basal medium) is preferably a protein- and serum-free culture medium. Said protein- and serum-free culture medium can be a commercially available medium or a chemically defined medium. Said culture medium may comprise an initial amount of cysteine (and/or cystine), tyrosine and tryptophan. Should said culture medium not comprise an initial amount of cysteine (and/or cystine), tyrosine and tryptophan, an initial amount of cysteine (and/or cystine), tyrosine and tryptophan can be added before or at the start of the culture in the bioreactor.

In the context of the invention as a whole, the main feed medium can be a standard main feed medium or concentrated main feed medium, such as a concentrated defined main feed medium. Preferably this main feed medium does not comprise Cys (neither cysteine nor cystine), Trp and Tyr.

In the context of the invention as a whole, the mammalian cells expressing the recombinant proteins are preferably cultivated in a fed batch process. In an embodiment, the production phase has a duration of at least 7 days, preferably at least 10 days, more preferably at least 12 days, such as 12 days, 13 days, 14 or 15 days. Although the addition of Cys, Trp and Tyr in the exceptional bolus is performed according to the invention, the culture can be supplemented at regular interval (such as daily or every other day) or on demand, with feed(s) (such as the main feed) comprising the remaining needed nutrients (e.g. amino acids other than Cys, Trp and Tyr, salts, sugar) until 1 or 2 days before the harvest.

In the context of the invention as a whole, the step of culturing said mammalian cells in a culture medium occurs preferably during a production phase.

In the context of the invention as a whole, the production phase is carried out in a bioreactor (such as a production bioreactor), preferably with a volume of equal or more than 50 L, equal or more than 100 L, equal or more than 500 L, equal or more than 1000 L, equal or more than 2,000 L, equal or more than 5,000 L, equal or more than 10,000 L or equal or more than 20,000 L. In other words, the mammalian cells producing the recombinant proteins are cultivated in a bioreactor (such as a production bioreactor), preferably with a volume of equal or more than 50 L, equal or more than 100 L, equal or more than 500 L, equal or more than 1000 L, equal or more than 2,000 L, equal or more than 5,000 L, equal or more than 10,000 L or equal or more than 20,000 L.

In the context of the invention as a whole, suitable mammalian host cells (also named mammalian cells) include Chinese Hamster Ovary (CHO cells), lymphocytic cell lines, e.g., NSO myeloma cells and SP2 cells, COS cells, myeloma or hybridoma cells. In a preferred embodiment, the mammalian cell is a CHO cell. Suitable types of CHO cells may include CHO-K1, CHOK1-SV, dhfr-CHO, such as CHO-DG44, CHO-DXB11, CHO-DXB1, or yet CHO-S cells. The host cells are preferably stably transformed or transfected with expression vectors encoding the recombinant protein of interest.

In the context of the invention as a whole, the recombinant protein is a protein such as a cytokine, a growth factor, a hormone, a fusion protein or an antibody. Should the protein be an antibody, it can be for instance a chimeric antibody, a humanised antibody or a fully human antibody and is preferably IgGs such as IgG1, IgG2, IgG3 or IgG4. Alternatively, it can be any kind as per the definition herein given. Preferably, the protein according to the methods, uses and processes of the present invention is an antibody or antigen-binding fragment thereof or a fusion protein. The processes according to the invention can further comprise the step of recovering the cell culture fluid (CCF) comprising the recombinant protein (harvest step). Subsequently to the harvest, the recombinant protein may be purified, e.g. if the protein is an antibody, using Protein A chromatography and other chromatographic/filtration steps. The processes further optionally comprise a step of formulating the purified recombinant protein, e.g. into a formulation with a high protein concentration, such as a concentration of 10 mg/ml or more, e.g. 50 mg/ml or more, such as 100 mg/ml or more, 150 mg/ml or more or yet 200 mg/mL or more. Without any limitation, the formulation can be a liquid formulation, lyophilised formulation or a spray-dried formulation.

In another embodiment, as the initial culture medium (or the basal medium) can be depleted of Cys, Tyr and Trp, the concentrations of Cys, Tyr and Trp provided in the cell culture by the exceptional bolus may consist of at least about 2.45 mM for Cys; at least about 1.50 mM for Trp; and at least about 2.75 mM for Tyr. Alternatively, the concentrations of Cys, Tyr and Trp provided in the cell culture by the exceptional bolus may consist of from about 2.45 mM to about 6.6 mM for Cys; from about 1.50 mM to about 2.9 mM for Trp; and from about 2.75 mM to about 6.2 mM for Tyr. In a further embodiment, should the initial culture medium (or the basal medium) contain Cys, Tyr and Trp, as the cells may consume very quickly these three amino acids, the concentrations of Cys, Tyr and Trp provided in the cell culture by the exceptional bolus may consist of: at least about 2.45 mM for Cys; at least about 1.50 mM for Trp; and at least about 2.75 mM for Tyr. Alternatively, the concentrations of Cys, Tyr and Trp provided by the exceptional bolus may consist of from about 2.45 mM to about 6.6 mM for Cys; from about 1.50 mM to about 2.9 mM for Trp; and from about 2.75 mM to about 6.2 mM for Tyr. Therefore, herein provided are processes for producing a recombinant protein, for culturing mammalian cells expressing a recombinant protein or for increasing the specific productivity of mammalian cells in culture, comprise the steps of culturing said mammalian cells in a culture medium and adding between day 1 and day 7 of the culture an exceptional bolus of cysteine (Cys), tryptophan (Trp) and tyrosine (Tyr), wherein the concentrations of Cys, Tyr and Trp provided in the cell culture by the exceptional bolus consist of: at least about 2.45 mM for Cys; at least about 1.50 mM for Trp; and at least about 2.75 mM for Tyr. Alternatively, herein provided are processes for producing a recombinant protein, for culturing mammalian cells expressing a recombinant protein or for increasing the specific productivity of mammalian cells in culture, comprise the steps of culturing said mammalian cells in a culture medium and adding between day 1 and day 7 of the culture an exceptional bolus of cysteine (Cys), tryptophan (Trp) and tyrosine (Tyr), wherein the concentrations of Cys, Tyr and Trp provided in the cell culture by the exceptional bolus consist of: from about 2.45 mM to about 6.6 mM for Cys; from about 1.50 mM to about 2.9 mM for Trp; and from about 2.75 mM to about 6.2 mM for Tyr.

### DESCRIPTION OF THE FIGURES

FIGS. 1A-1B: FIG. 1A) Preparation of a “normal” feed from powder. FIG. 1B) Preparation of a “concentrated” feed from powder.

FIG. 2: Standard feeding strategy versus feeding strategy according to the invention. Each pick corresponds to a bolus addition of Cys, Tyr and Trp.

FIG. 3: Viable Cell Concentration profiles for CHO cell line producing Mab-1.

FIG. 4: Mab-1 titre profiles.

FIG. 5: Specific productivity profiles.

FIG. 6: Main charges for mAb-1.

FIG. 7: Drug colour intensity for mAb-1.

FIG. 8: Specific productivity contour plots.

FIG. 9: Cumulative Integral Viable Cell Count contour plots for CHO cell line producing Mab-2.

FIG. 10: Specific productivity for mAb-2.

FIGS. 11A-11C: FIG. 11A) VCC for CHO cell line producing Mab-3, FIG. 11B) Titre of mAb-3 and FIG. 11C) Specific productivity of cell line producing Mab-3.

FIG. 12: Characterisation profiles of mAb3 (acidic species, main species and basic species)

FIG. 13: Specific productivity for mAb-4.

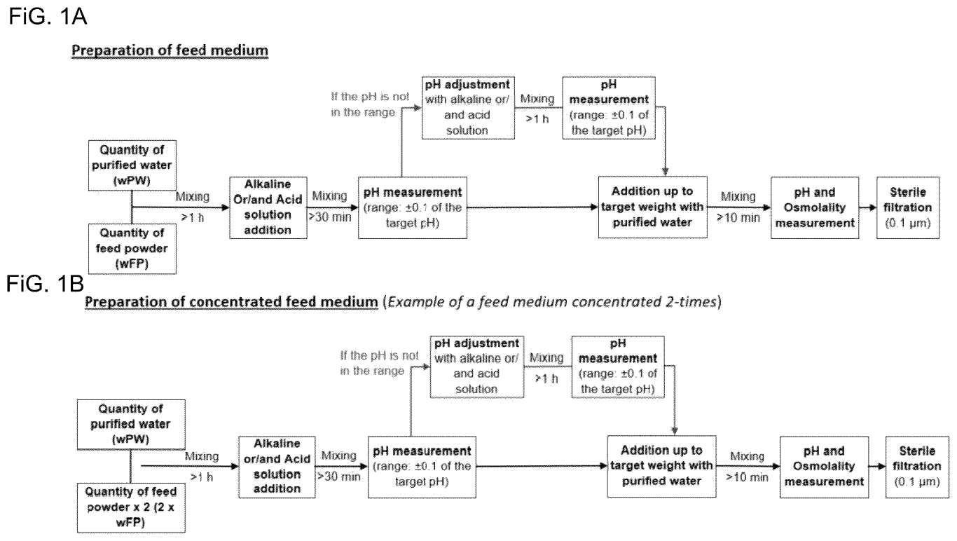

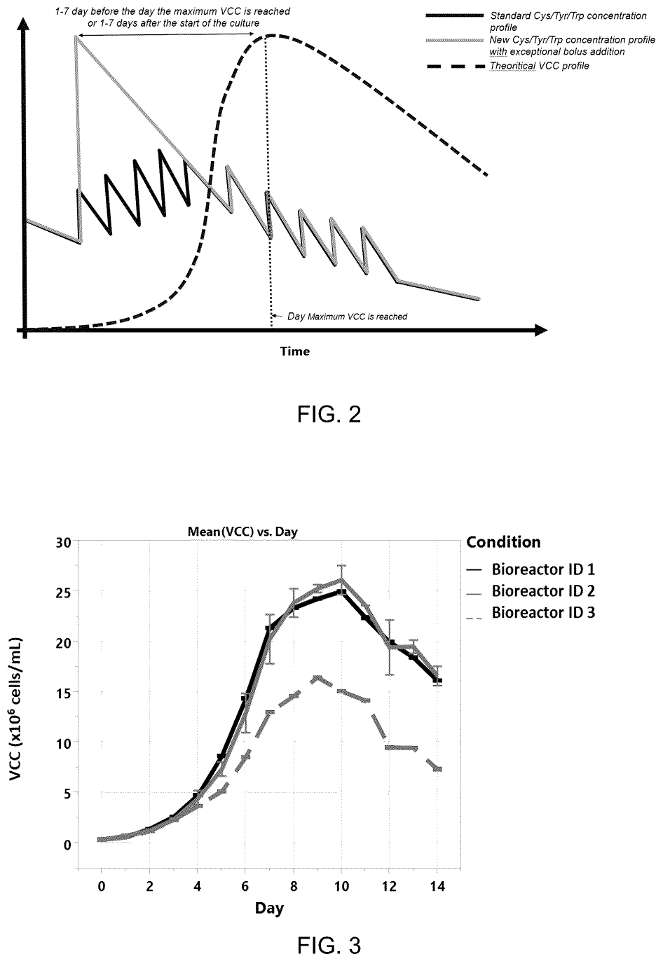

### EXAMPLES

### Cell Line, Cell Culture and Experimental Procedure

Four different production CHO-DG44 cell lines were used, respectively producing: mAb-1 (a full IgG4 antibody having a pl of 7.3-7.95), mAb-2 (a Trybe antibody having a pl of 8.6-9.2), mAb-3 (a full IgG4 antibody having a pl of 8.1-8.4), mAb-4 (a full IgG4 antibody having a pl of 6.1-6.3).

The cells were cultivated in 2 L stirred tank glass bioreactor (STR) with supply towers (C-DCUII, Sartorius Stedim Biotech) controlled by a multi-fermentation control system (MFCS, Sartorius Stedim Biotech) or in shake flasks. The reactors were equipped with a 3-segment blade impeller. The cultivation start volume was adapted to ensure the cultivation end volume is optimal. The production bioreactors were seeded at a target seeding density (TSD). The pH control of the production bioreactor was controlled to about 7.0. To control pO2 in a specified range, air, nitrogen and oxygen were sparged into the culture vessel based on a cascade controller using a predefined mixture profile. The temperature was controlled at about 37° C. The production was operated in fed-batch mode for 14 days. During this phase, the monoclonal antibody (mAb) is secreted into the medium. Samples were drawn daily to determine VCC, viability, off-line pH, pCO2, osmolality, glucose-lactate concentration, amino acid concentration and mAb concentration (stocked at −80° C.). Antifoam was added manually on demand. 72 hours after inoculation, continuous nutrient feeding was started with a predetermined rate with a feed medium 1 (main feed; comprising the needed nutrients except Cys, Tyr and Trp). Glucose (feed medium 2) was added to the culture on demand when the glucose concentration dropped below a certain threshold (daily measurement). Except mentioned otherwise, the feed medium 3 containing Cys, Tyr and Trp was added as a daily bolus starting either from day 3 (for the bioreactors inoculated at 0.35×10⁶ cells/mL) or from day 0 (for the bioreactors inoculated at higher cells density) until day 12 of the production. Samples for the amino acid analysis were taken before the feed medium 3 addition. The extracellular concentrations after feeding were computed based on the composition of feed medium 3 and measured nutrients concentration before feed medium 3 addition or on the basal medium composition.

### Analytical Methods

Cells were counted using a VI-CELL® XR (Beckman-Coulter) automated cell counting device that operated based on trypan blue exclusion. Glucose and lactate levels in the culture medium were determined using a NOVA 400 BioProfile automated analyzer (Nova Biomedical) or a Cedex Bio HT (Roche). Metabolites concentrations were also determined daily using a CedexBioHT system (Roche). Product titres analysis were performed with a ForteBio Octet model analyzer (ForteBio Inc) or with the CEDEX or protein A high-pressure liquid chromatography (HPLC) with cell culture supernatant samples which were stored at −80° C. prior to analysis. Amino acids were analysed by reversed-phase UPLC (Waters AccQ Tagultra method) after ultra-filtration using Amicon Ultra-0.5 mL centrifugal filters (Merck Millipore). The cell culture supernatant samples were harvest and purified with a Protein A purification on the ÄKTA Xpress system. The relative percentage of main, acidic (APG for Acidic Peak Group) and basic (BPG for Basic Peak Group) isoform of the purified mAb was determined by Imaged Capillary Electrophoresis (ProteinSimple iCE3). Aggregate (HMWS), monomer and fragment (LMWS) levels of the purified mAb were determined by size exclusion chromatography (SE-UPLC) or protein A HPLC gradient. Colour intensity of the concentrated antibody composition was measured in the concentrated protein A eluates using a spectrophotometer by transmission (UltrascanPro) and compared to the CIE (Commission Internationale de l'Eclairage) scale. The numerical results were normalized to the concentration of 40 mg/mL. Statistical analyses were performed using SAS software JMP 110.

### Example 1

For this experiment 2 L bioreactors were inoculated with CHO cells producing mAb-1 at a seeding density of 0.35×10⁶ cells/mL. Three conditions were tested in fed-batch process as described in the experimental procedure. Bioreactor (BR) ID 1 and 2 had the same feeding strategy (Cys, Trp and Tyr were added every day, with the first bolus addition on day 3) but in BR ID 2, the concentrations of Cys, Tyr and Trp were twice lower. BR ID 3 had the same feed medium composition than condition 2 (i.e. the concentrations of Cys, Tyr and Trp were twice lower compared to BR ID 1). However instead of a daily feed of Cys, Trp and Tyr, starting at day 3, the feed containing Cys, Tyr and Trp was added on day 3 as an exceptional bolus (alternatively named high bolus) corresponding to the quantity of Cys, Tyr and Trp that should have been added between day 3 and 7 of Bioreactor ID 2. After day 3 and until day 7 included, Cys, Tyr and Trp were no longer added with the feed medium into the cell culture production of Bioreactor ID 3. As of day 8, the feeding strategy is similar to Bioreactor ID 2 (i.e. feeding of Cys, Trp and Tyr resumes). The objective was to assess the impact of high concentration of Cys, Tyr, Trp and of the timing of addition on cell growth and specific productivity of the recombinant mAb-1 (the max VCC for this cell line expressing mAb-1 was estimated at day 10 from preliminary experiments, and confirmed in each of the bioreactor run in this example with BR ID 1 and 2, i.e. in the conditions without exceptional bolus).

TABLE 1
Experimental conditions

| | | | | | Max. Cys | Max Trp | Max Tyr |
|---|---|---|---|---|---|---|---|
| | Cys cc | Trp cc | Tyr cc | | cc before | cc before | cc before |
| BR | in Feed | in Feed | in Feed | Exceptional | day 10 | day 10 | day 10 |
| (ID) | (%) | (%) | (%) | Bolus | (g/L) ^{(a)} | (g/L) ^{(a)} | (g/L) ^{(a)} |
| 1 | 100% | 100% | 100% | No | 0.25 | 0.26 | 0.34 |
| 2 | 50% | 50% | 50% | No | 0.13 | 0.20 | 0.25 |
| 3 | 50% | 50% | 50% | Yes | 0.34 | 0.37 | 0.98 |

^{(a)} Estimated maximum concentration of the amino acid after exceptional bolus addition based on the amount of amino acid measured in the bioreactor before addition of the exceptional bolus and the quantity of said amino acid brought by the feed.

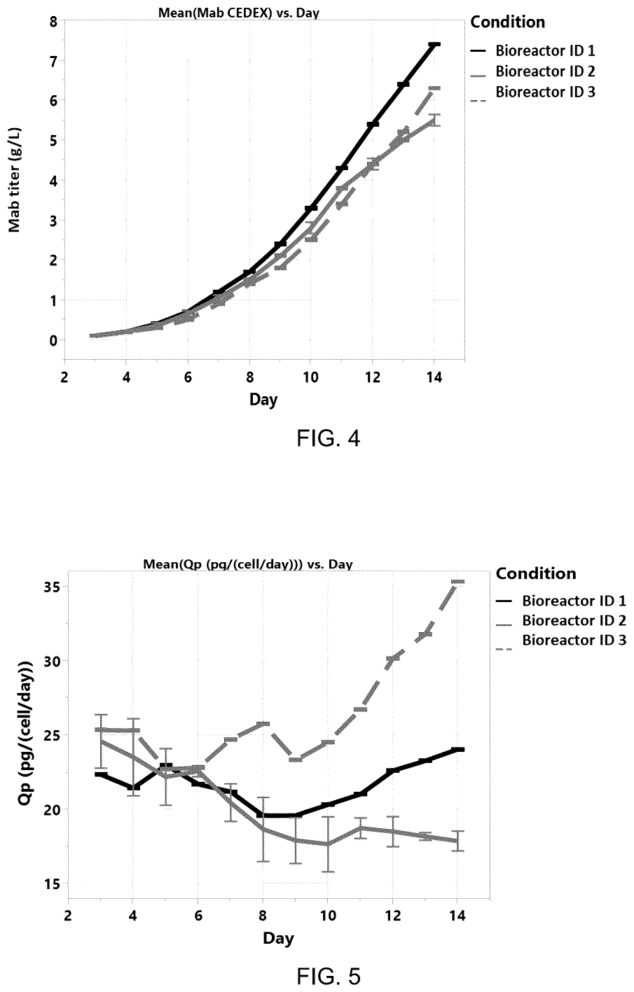

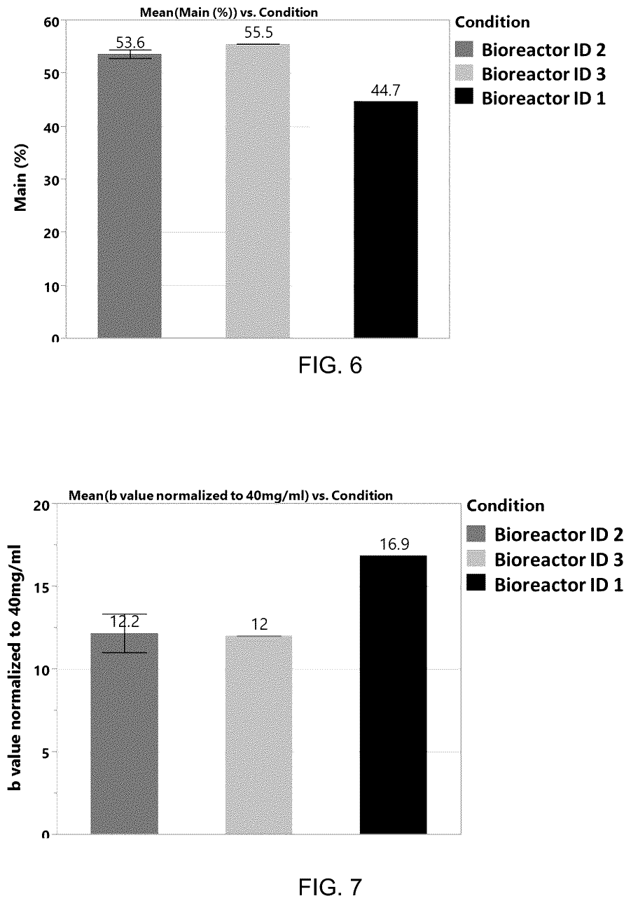

The results shown in FIG. 3 demonstrate a lower cell growth for the condition with high concentration of Cysteine, Tyrosine and Tryptophan reached (in the cell culture) before maximum VCC was reached (BR ID 3; i.e. before day 8). Despite the low VCC profile for Bioreactor ID 3, the final titre for that condition was higher than Bioreactor ID 2 (see FIG. 4). From a specific productivity viewpoint (FIG. 5) it was shown that the Qp was significantly increased for Bioreactor ID 3. Finally, the main charges measured as described in experimental procedure section (FIG. 6), were equivalent between Bioreactor ID 2 and 3, confirming that there is no impact of high concentration of Cys, Tyr and Trp on recombinant protein charge variants if the total quantities of Cys, Tyr and Trp added throughout the production per total amount of mAb produced were unvaried. Similar trend was observed with colour intensity (FIG. 7). Further, although the total amounts of Cys, Tyr and Trp were identical for BR ID2 and BR ID3, the addition of said amount at once, as an exceptional bolus and according to a particular timing, had a positive impact on inhibiting cell growth while increasing specific productivity.

Conclusion of example 1: The addition at once (simultaneously or concomitantly), and according to a particular timing, of high amounts of Cys, Tyr and Trp inhibited cell growth and increased specific productivity (mAb1). Indeed, from the experiments presented in this example, it was concluded that high concentrations of Cys, Tyr and Trp, i.e. at least 0.34 g/L, 0.37 g/L and 0.98 g/L, reached (in the cell culture) during cell culture production before the day of maximum VCC, led to an increase in specific productivity.

### Example 2

For this experiment, 15×2 L bioreactors were inoculated with CHO cells producing mAb-2 at a seeding density of 3.75×10⁶ cells/mL in fed-batch process as described in the experimental procedure, above. In this experiment, multiple conditions with various maximum concentrations of Cys, Tyr and Trp reached (in the cell culture) at different point in time before the day of maximum VCC were tested (Table 2). For all conditions, the same total quantity (total quantity brought via the basal medium as well as via the different feeds) of Cys, Tyr and Trp were added throughout the production run. The max VCC for the cell line expressing mAb-2 was estimated at day 7 in preliminary experiments and was confirmed for each bioreactor run in this example.

TABLE 2
Experimental conditions.

| | | Day of | | | | Volume of feed |
|---|---|---|---|---|---|---|
| | | bolus | Max. Cys | Max Trp | Max Tyr | added during |
| | | addition | cc after | cc after | cc after | exceptional |
| | | prior day of | bolus | bolus | bolus | bolus addition |
| BR | Exceptional | maximum | addition | addition | addition | (A < B < C < |
| (ID) | Bolus? | VCC | (g/L) ^{(a)} | (g/L) ^{(a)} | (g/L) ^{(a)} | D < E < F < G) |
| 4 | No | — | 0.06 | 0.20 | 0.20 | — |
| 5 | Yes | 0 | 0.36 | 0.35 | 0.55 | B |
| 6 | Yes | 4 | 0.74 | 0.55 | 1.02 | F |
| 7 | Yes | 2 | 0.32 | 0.33 | 0.51 | A |
| 8 | No | — | 0.06 | 0.20 | 0.20 | — |
| 9 | Yes | 4 | 0.45 | 0.40 | 0.66 | C |
| 10 | Yes | 0 | 0.56 | 0.45 | 0.80 | D |
| 11 | Yes | 2 | 0.62 | 0.48 | 0.86 | E |
| 12 | Yes | 0 | 0.72 | 0.53 | 0.99 | F |
| 13 | Yes | 0 | 0.36 | 0.35 | 0.55 | B |
| 14 | Yes | 4 | 0.74 | 0.55 | 1.02 | F |
| 15 | No | — | 0.06 | 0.20 | 0.20 | — |
| 16 | Yes | 4 | 0.45 | 0.4 | 0.66 | C |
| 17 | Yes | 0 | 0.56 | 0.45 | 0.8 | D |
| 18 | Yes | 2 | 0.82 | 0.59 | 1.11 | G |

^{(a)} Estimated maximum concentration of the amino acid after exceptional bolus addition based on the amount of amino acid measured in the bioreactor before addition of the exceptional bolus and the quantity of said amino acid brought by the feed.

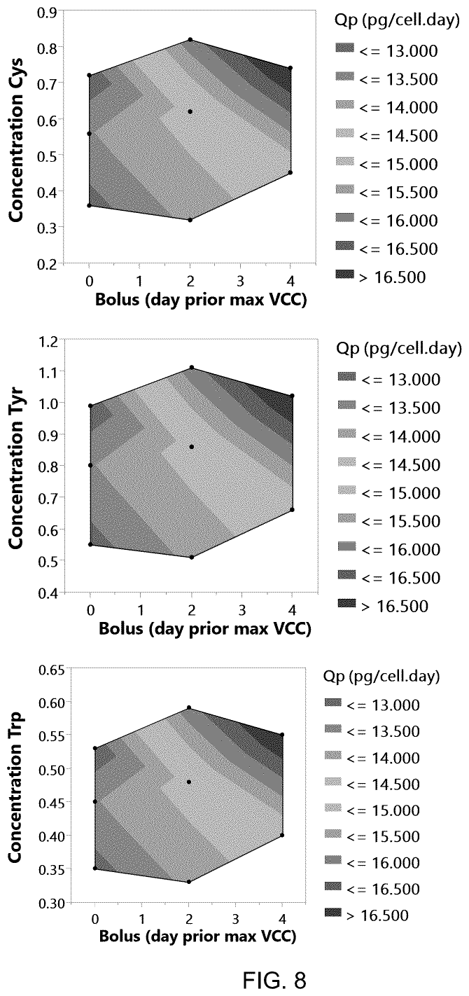

The specific productivity (FIG. 8) was increased when the exceptional bolus of Cysteine, Tryptophan and Tyrosine were added before reaching the maximum VCC (i.e. 2 or 4 days before) for the highest concentration reached (in the cell culture) after the exceptional bolus for concentration higher or equal to 0.32 g/L, 0.33 g/L and 0.51 g/L respectively. No impact on specific productivity was observed when high concentration of Cysteine, Tryptophan and Tyrosine were reached (in the cell culture) on day of maximum VCC.

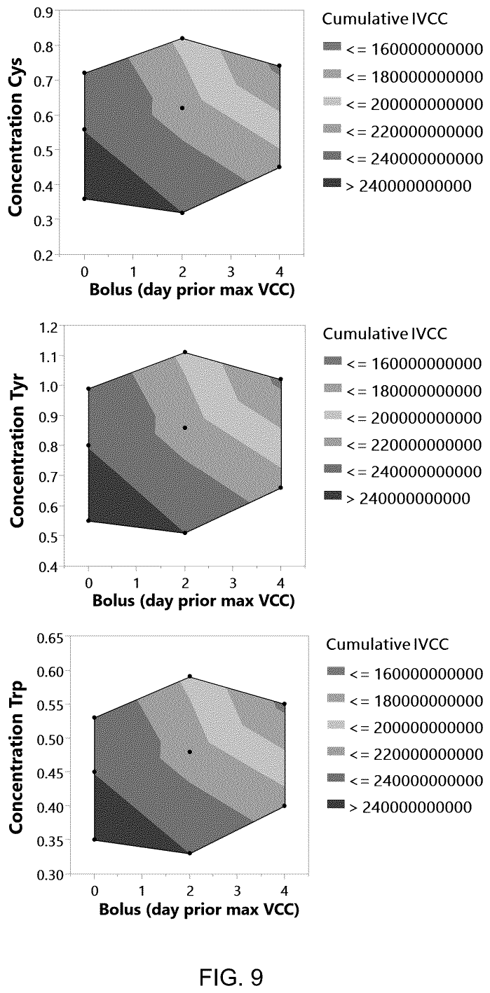

The cumulative IVCC (FIG. 9) was also reduced when the exceptional bolus of Cysteine, Tryptophan and Tyrosine were performed 2 or 4 days before the day of maximum VCC for the highest concentration reached (in the cell culture) after the high bolus, i.e. for concentration higher or equal to 0.32 g/L, 0.33 g/L and 0.51 g/L respectively.

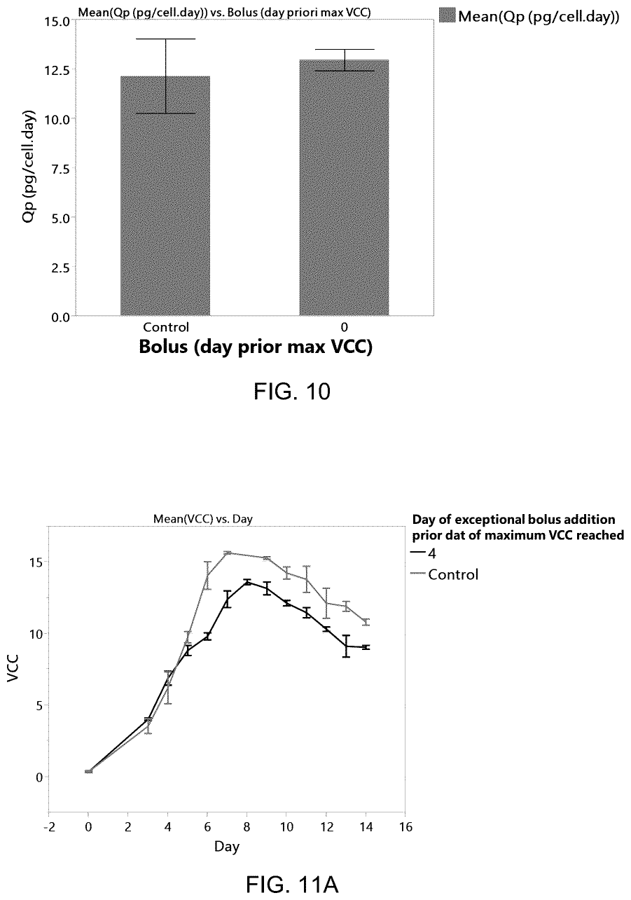

When the specific productivity of the control condition with low concentration of Cys, Tyr and Trp reached (in the cell culture) before day of maximum VCC (Control condition) and conditions with high concentrations of Cys, Tyr and Trp reached (in the cell culture) the day of maximum VCC achieved were compared, no significant differences were observed (FIG. 10).

Conclusion of example 2: The addition, in a timely manner, of an exceptional bolus of Cys, Tyr and Trp (leading to a high total concentration of Cys, Tyr and Trp in the cell culture medium) inhibited cell growth and increased specific productivity (mAb2). The benefits of the addition of this exceptional bolus of Cys, Trp and Tyr (leading to high total concentration of Cys, Tyr and Trp in the cell culture medium) on Qp seem to be observed only when said addition, in the cell culture, is performed before the day of maximum VCC is reached. High total concentrations of Cys, Tyr and Trp have to be reached simultaneously or concomitantly in the cell culture medium.

### Example 3

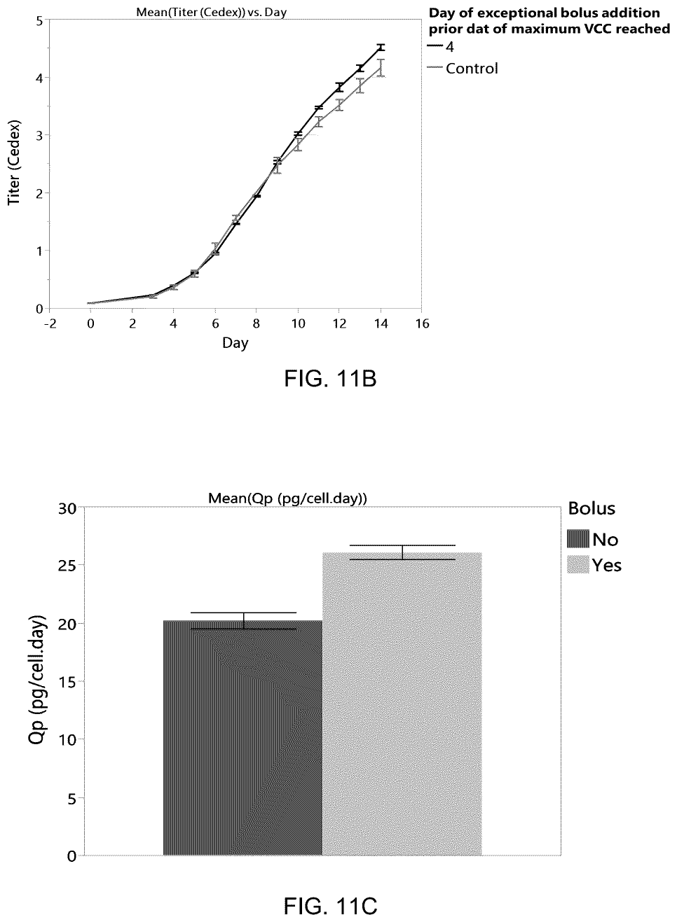

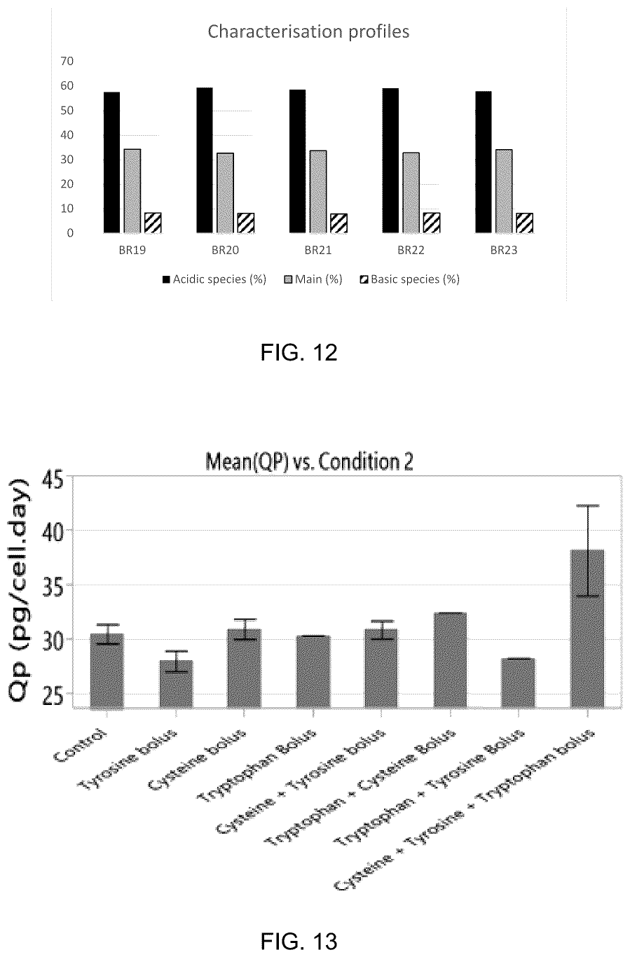

For this experiment, 5×2 L bioreactors were inoculated with CHO cells producing a full antibody, (mAb-3) at a seeding density of 3.75×10⁶ cells/mL in fed-batch process as described in the experimental procedure, above. In this experiment, two conditions with various maximum concentrations of Cys, Tyr and Trp reached (in the cell culture) 4 days before the day of maximum VCC was reached (max VCC was observed at day 8 for this cell line) were tested (Table 3). For all conditions, the same total quantities (total quantity brought via the basal medium as well as via the different feeds) of Cys, Tyr and Trp were added throughout the production run. The average specific productivity depicted in FIG. 11C show that the bioreactors with the highest concentration of Cys, Tyr and Trp reached during the production, i.e. 0.80 g/L, 1.08 g/L and 0.58 g/L respectively (BR ID 19 & BR ID 20), led to the highest specific productivity. This example confirmed what was observed with other cell lines producing other mAbs, that high concentration of Cys, Tyr and Trp (in the cell culture) during cell culture production before the day on which maximum VCC is reached, led to an increase of specific productivity. These high concentrations were reached thanks to an exceptional bolus added in a timely manner. As shown in FIG. 12, the characterization profile of mAb3 (Neutral form, acidic species and basic species) is similar (less than 1 point of variation) whatever the condition. Therefore, there is no impact of the timing of the high bolus on the quality of the antibody (except that it should be performed at least one day before the VCC is reached).

TABLE 3
Experimental conditions

| | | Day of bolus | | | |
|---|---|---|---|---|---|
| | | addition prior | | | |
| | | day of | Max. Cys cc | Max Trp cc | Max Tyr cc |
| BR | Exceptional | maximum | after bolus | after bolus | after bolus |
| (ID) | Bolus | VCC reached | addition (g/L) ^{(a)} | addition (g/L) ^{(a)} | addition (g/L) ^{(a)} |
| 19 | Yes | 4 | 0.80 | 0.58 | 1.08 |
| 20 | Yes | 4 | 0.80 | 0.58 | 1.08 |
| 21 | No | Control | 0.06 | 0.2 | 0.2 |
| 22 | No | Control | 0.06 | 0.2 | 0.2 |
| 23 | No | Control | 0.06 | 0.2 | 0.2 |

^{(a)} Estimated maximum concentration of the amino acid after exceptional bolus addition based on the amount of amino acid measured in the bioreactor before addition of the exceptional bolus and the quantity of said amino acid brought by the feed.

Conclusion of example 3: Once more, this example confirmed the positive impact of reaching high total concentrations of Cys, Tyr and Trp in the cell culture (thanks to the addition of an exceptional bolus if Cys, Trp and Tyr), before the day of maximum VCC was reached, on increasing specific productivity.

### Example 4—Synergistic Effect of the Three Amino Acids

This experiment was designed to assess whether the impact of high concentrations of Cys, Tyr and Trp (in the cell culture) on specific productivity was due to a synergistic effect of the three amino acids or if it was due to only one or two amino acids.

For this example, 19 shake flasks were inoculated with CHO cells producing mAb-4 at a seeding density of 0.35×10⁶ cells/mL in fed-batch process as described in the materials and methods. Multiple conditions with various maximum concentration of Cys, Tyr and Trp reached 4 days before the day of maximum VCC was reached (i.e. day 8) were tested (Table 4) and the effect of the three amino acids was decoupled. For all conditions, the same total quantity (total quantity brought via the basal medium as well as via the different feeds) of Cys, Tyr and Trp were added throughout the production run. The maximum concentrations reached for Cys, Tyr and Trp (in the cell culture) were 0.48 g/L, 0.71 g/L and 0.41 g/L respectively, just after addition of the exceptional bolus.

The average specific productivity for all conditions depicted in FIG. 13 shown that a surprising synergistic increase in specific productivity was obtained with the combination of high concentrations of Cys, Tyr and Trp.

Conclusion: This example confirmed, yet with another cell line (expressing antibody mAb-4), that when high total concentrations of Cys, Tyr and Trp are present in the cell culture before the day the maximum VCC (i.e. at least at least about 2.45 mM for Cys; at least about 1.50 mM for Trp and at least about 2.75 mM for Tyr; thanks to an exceptional bolus addition), this leads to an increase of specific productivity (Qp). High total concentrations of Cys, Tyr and Trp have to be reached simultaneously or concomitantly. The effect of the simultaneously or concomitantly high concentration of Cys, Tyr and Trp was synergistic and not additive.

TABLE 4
Experimental conditions

| | | Day of bolus | | | |
|---|---|---|---|---|---|
| | | addition prior | Max. Cys cc | Max Trp cc | Max Tyr cc |
| | | day of | after bolus | after bolus | after bolus |
| BR | Exceptional | maximum VCC | addition | addition | addition |
| (ID) | Bolus | reached | (g/L)^{(a)} | (g/L) ^{(a)} | (g/L) ^{(a)} |
| 24 | Cys + Tyr + Trp | 4 | 0.48 | 0.41 | 0.71 |
| 25 | Cys + Tyr + Trp | 4 | 0.48 | 0.41 | 0.71 |
| 26 | No | Control | 0.06 | 0.2 | 0.2 |
| 27 | Cys + Tyr | 4 | 0.48 | 0.2 | 0.71 |
| 28 | Cys + Tyr | 4 | 0.48 | 0.2 | 0.71 |
| 29 | Cys | 4 | 0.48 | 0.2 | 0.2 |
| 30 | Cys | 4 | 0.48 | 0.2 | 0.2 |
| 31 | Tyr | 4 | 0.06 | 0.2 | 0.71 |
| 32 | Tyr | 4 | 0.06 | 0.2 | 0.71 |
| 33 | No | Control | 0.06 | 0.2 | 0.2 |
| 34 | Trp + Tyr | 4 | 0.06 | 0.41 | 0.71 |
| 35 | Cys + Tyr | 4 | 0.48 | 0.2 | 0.71 |
| 36 | Cys | 4 | 0.48 | 0.2 | 0.2 |
| 37 | Trp | 4 | 0.06 | 0.41 | 0.2 |
| 38 | No | Control | 0.06 | 0.2 | 0.2 |
| 39 | Trp + Cys | 4 | 0.48 | 0.41 | 0.2 |
| 40 | Tyr | 4 | 0.06 | 0.2 | 0.71 |
| 41 | Cys + Tyr | 4 | 0.48 | 0.2 | 0.71 |
| 42 | Cys | 4 | 0.48 | 0.2 | 0.2 |

### REFERENCES

1) Hecklau C et al. (2016) J Biotech, 218:53-63
2) Zang Li. et al. (2011) Anal. Chem, 83:5422-5430
3) Purdie J. et al. (2016) Biotechnology Progress, 32:998-1008
4) Banks D. D. et al. (2009), Journal of pharmaceutical sciences, 98:4501-4510
5) Kshirsagar R. et al. (2012) Biotechnology and Bioengineering, 109 (10): 2523-2532
6) US2013/0281355
7) WO2013/158275
8) Ben Yahia et al. (2015) Appl Microbiol Biotechnol, 99:7009-7024
9) Ben Yahia et al. (2016) Biotechnology and Bioengineering, 114:785-79
10) WO2018/219968
11) Pan et al. (2017) Cytotechnology, 69:39-56
12) U.S. Pat. No. 8,765,413
13) Ozturk & Hu (2005) Cell Culture Technology for Pharmaceutical and Cell-Based Therapies, ed. CRC Press.
14) WO98/08934
15) US2006/0148074
16) Voisard et al. (2003) Biotechnology and Bioengineering, 82 (7): 751-765
17) WO2019/106091
18) WO2015/197772

1-19. (canceled)

20. A process for producing a recombinant protein, wherein the process comprises the steps of culturing mammalian cells expressing said recombinant protein in a culture medium and adding, between day 1 and day 7 of the culture, an exceptional bolus of cysteine (Cys), tryptophan (Trp) and tyrosine (Tyr), wherein said exceptional bolus provides high concentrations of Cys, Tyr and Trp in the cell culture.

21. A process for increasing specific productivity (Qp) of mammalian cells in culture, wherein the mammalian cells express a recombinant protein, comprises the steps of culturing the mammalian cells in a culture medium and adding between day 1 and day 7 of the culture an exceptional bolus of (Cys), tryptophan (Trp) and tyrosine (Tyr), wherein said exceptional bolus provides high concentrations of Cys, Tyr and Trp in the cell culture.

22. The process according to claim 20, wherein the total concentrations of Cys, Trp and Tyr present in the cell culture upon addition of the exceptional bolus are respectively:
a) at least about 2.45 mM for Cys;
b) at least about 1.50 mM for Trp; and
c) at least about 2.75 mM for Tyr.

23. The process according to claim 20, wherein the concentrations of Cys, Tyr and Trp of the exceptional bolus are adapted in order to reach high total concentrations of Cys, Tyr and Trp in the cell culture, wherein the high concentrations of Cys, Tyr and Trp to be reached consist of:
a) at least about 2.45 mM for Cys;
b) at least about 1.50 mM for Trp; and
c) at least about 2.75 mM for Tyr.

24. The process according to claim 20, further comprising the preliminary step of performing at least one initial experiment to determine the day the maximum viable cell concentration (VCC) is reached for said mammalian cells and/or the preliminary step of performing at least one initial experiment to determine the daily concentrations of Cys, Trp and Tyr in the culture medium after the start of the culture, in order to determine the amounts of Cys, Trp and Tyr to be added via the exceptional bolus.

25. The process according to claim 24, wherein the addition of the exceptional bolus of Cys, Tyr and Trp in the cell culture is performed before the day the maximum viable cell concentration (VCC) is reached.

26. The process according to claim 24, wherein the addition of the exceptional bolus of Cys, Tyr and Trp in the cell culture is performed at least one day before the maximum VCC is reached.

27. The process according to claim 24, wherein the total concentrations of Cys, Trp and Tyr present in the cell culture upon addition of the exceptional bolus are at least 2.45 mM, 1.50 mM and 2.75 mM, respectively, and wherein said total concentrations are reached at least one day before the maximum VCC is reached.

28. The process according to claim 24, wherein the addition of the exceptional bolus of Cys, Tyr and Trp in the cell culture is performed 1 to 7 day(s) before the day the maximum VCC is reached.

29. The process according to claim 20, wherein Cys, Tyr and Trp, in the exceptional bolus, are added:
(i) simultaneously, either:
(a) combined in a single solution,
(b) in individual solutions each comprising one of Cys, Tyr and Trp; or
(c) a combination of two amino acids in one solution and a second solution comprising the remaining amino acid; or
(ii) sequentially, in any order.

30. The process according to claim 20, wherein the step of culturing said mammalian cells in a culture medium occurs during a production phase.

31. The process according to claim 20, wherein the cell culture is performed according to a fed batch process.

32. The process according to claim 20, wherein further additions of Cys, Tyr and Trp are performed, after the exceptional bolus addition, throughout the culture, in order to prevent any depletion Cys, Tyr and Trp.

33. The process according to claim 20, further comprising the step of adding a main feed medium to the cell culture.

34. The process according to claim 33, wherein the main feed medium does not comprise Cys, Tyr or Trp.

35. The process according to claim 20, wherein the mammalian cells are Chinese Hamster Ovary (CHO) cells.

36. The process according to claim 20, wherein the recombinant protein is a cytokine, a growth factor, a hormone, an antibody or a fusion protein.

37. The process according to claim 20, wherein the high concentrations of Cys, Tyr and Trp provided in the cell culture by the exceptional bolus consists of:
a) at least about 2.45 mM for Cys;
b) at least about 1.50 mM for Trp; and
c) at least about 2.75 mM for Tyr.

38. A process for culturing mammalian cells expressing a recombinant protein, comprising the steps of culturing said mammalian cells in a culture medium and adding between day 1 and day 7 of the culture an exceptional bolus of cysteine (Cys), tryptophan (Trp) and tyrosine (Tyr), wherein said exceptional bolus provides high concentrations of Cys, Tyr and Trp in the cell culture.
